(19) United States
(12) Patent Application Publication
(10) Pub. No.: US 2010/0233244 A1
(43) Pub. Date: Sep. 16, 2010

(54) Smoking Withdrawal Combination Wafer

(75) Inventors: Hans-Rainer Hoffmann, Neuwied (DE); Reto Brändli, San Francisco, CA (US); Frank Theobald, Wehr (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andernach (DE)

(21) Appl. No.: 12/308,428

(22) PCT Filed: Jun. 4, 2007
(86) PCT No.: PCT/EP2007/004937

§ 371 Date: Jan. 22, 2009

(30) Foreign Application Priority Data
Jun. 16, 2006 (DE) 10 2006 027 795.3

(51) Int. Cl. A61K 9/70; A61K 31/551; A61K 31/465; A61K 31/5513; A61K 31/55; A61K 31/5375; A61K 31/5415; A61K 31/382; A61K 31/404; A61K 31/122; A61K 31/519; A61K 31/4375; A61K 31/445; A61K 31/225; A61P 25/34; B05D 7/00

(52) U.S. Cl. 424/443; 514/220; 514/343; 514/221; 514/214.02; 514/239.2; 514/226.2; 514/437; 514/415; 514/680; 514/225.8; 514/259.41; 514/280; 514/317; 514/547; 427/2.14

(57) ABSTRACT

The present invention relates to a quickly decomposing oral drug preparation, for the application of active ingredient combinations for smoking withdrawal, which contains nicotine, a nicotine salt, a nicotine derivative, or a substance that reacts to nicotine, in combination with another active ingredient, and the use of such a drug preparation for the treatment of smoking withdrawal, and the use of nicotine, and/or nicotine salts or derivatives, for the production of medications for the treatment of smoking withdrawal. The active ingredient that is to be administered, in combination, for this purpose is a centrally active ingredient, preferably an antidepressant for the fighting of psychic dependency in terms of a smoking withdrawal therapy. The administration of the active ingredient combination to the patient should be handled in a simple and reliable way and should exclude side effects to a large extent.

### CROSS-REFERENCE TO RELATED APPLICATIONS

This application is a National Stage application of International Application No. PCT/EP2007/004937, filed on Jun. 4, 2007, which claims priority of German application number 10 2006 027 795.3, filed on Jun. 16, 2006, both of which are incorporated herein by reference in their entireties.

### BACKGROUND OF THE INVENTION

1. Field of the Invention

The present invention relates to quickly decomposing oral dosage forms for the application of active agent combinations for smoking withdrawal, which contain nicotine, a nicotine salt, a nicotine derivative, or a substance with nicotinergic action, in combination with another active agent.

The invention further relates to the use of such dosage forms for the treatment of nicotine dependence, for nicotine replacement or for smoking withdrawal, and the use of nicotine, and/or nicotine salts or derivatives, for the production of pharmaceutical forms for the treatment of nicotine dependence.

2. Description of the Prior Art

About 30% of the world population smoke and consume about 6 trillion cigarettes per year. Smoking, like the consumption of alcohol, is one of the socially accepted and wide-spread types of drug consumption, with the alkaloid nicotine, which mainly occurs in tobacco, having an effect comparable to that of other narcotics which leads to physical dependency. In smokers, the toxic effects of nicotine, which is a strong neurotoxin, are repressed by tolerance.

Already shortly after inhalation, nicotine enters the brain where it acts on acetylcholine receptors, triggering a number of physiological reactions. This leads to an increase in heart rate, a narrowing of the blood vessels with associated increase in blood pressure, and a marked decline in skin temperature. In addition, via central effects, nicotine increases psychomotoric performance as well as attention and memory performances.

The high addictive potential of nicotine is above all attributed—apart from the direct effect on the nicotinergic acetylcholine receptors—to its influence on the dopamine system, which is assumed to play the decisive role in the rewarding effect of smoking.

As regular nicotine consumption leads to an increase in the number of central nicotinergic acetylcholine receptors, a discontinuation of the nicotine supply causes withdrawal symptoms.

Apart from nicotine, more than 4,000 compounds contained in tobacco have been identified, of which many have a carcinogenic effect or are at least suspected of causing cancer.

Nicotine consumption is a major cause of vascular diseases, high blood pressure, cancer and asthma, and of the associated late sequelae such as apoplexy, cardiac infarction, chronic bronchitis, COPD (chronic obstructive pulmonary disease), smoker's leg, arteriosclerosis and impaired vision.

Statistics show that certain serious illnesses are directly attributable to smoking. Thus, 90% to 95% of lung cancer cases, 90% of amputations, and almost all cardiac infarctions prior to the age of 40 are related to smokers. Altogether, even 30% of all cancer cases are being attributed to cigarette consumption. It has furthermore turned out that the risk of thrombosis is 10 times higher in female smokers if they take oral contraceptives, whereas more recent studies are directed at showing that erectile dysfunction is considerably more frequent in smoking men.

Altogether, the life expectancy of smokers in Germany is approximately 10% below that of non-smokers, and almost one fourth of all “premature” deaths is due to sequelae of smoking.

In addition, the number of premature invalids due to smoking is estimated to be 70,000 to 100,000 per year, and the number of those dying from the consequences of “passive smoking” is estimated to be approximately 500 to 3,500.

According to estimates by the Deutsche Gesellschaft für Nikotinforschung, the total costs caused by smoking amount to approximately 75 billion Euro per year.

Because of the above-discussed negative consequences and the health hazards, smoking has increasingly entered the focus of the health policy discussion. Not least because it has meanwhile been proven that passive smoking, too, can lead to serious illnesses.

The space for smokers is increasingly being restricted, and in many public spaces and at the workplace smoking is largely prohibited. In the USA, Italy and Ireland the ban on smoking in restaurants and pubs has already been confirmed by corresponding legislation.

In addition, the costs for tobacco products in Germany have risen strongly in the past years, and smokers will face further costs, for instance through raised contributions to health insurance funds to cover the additional public health costs caused by smoking. Thus, tobacco consumption is increasingly turning into a luxury involving financial aspects that are not to be neglected. Given a price of about 20 cents per cigarette, a smoker smoking 20 cigarettes a day, for example, will burn about 1,500 Euro per year.

In light of the above-mentioned figures and of the known detrimental effects of tobacco smoking on health, there are therefore many good reasons for not smoking or to quit smoking, in addition to the obvious financial aspects.

Nevertheless, for most nicotine addicts, putting an end to dependence is possible only with great difficulty. The main reason for this is the withdrawal symptoms that occur after quitting tobacco consumption.

Quitting the addiction is therefore facilitated if at least during a detoxification phase the need for nicotine is satisfied in another way, for instance within the framework of a nicotine replacement therapy. This can be done, for example, by means of so-called nicotine patches that deliver nicotine to the human organism via the skin, thereby suppressing nicotine withdrawal symptoms so that smoking cessation is facilitated. However, a disadvantage of these transdermal therapeutic systems (TTS's) is that they remain on the skin over a long period of time and are felt to be disturbing. In unfavourable cases, irritation of the skin and allergic reactions can be caused by both nicotine and the adhesive. Moreover, by means of TTS's, nicotine is delivered continuously to the organism, but peak concentrations, such as they occur with smoking and which may be responsible for the reward effects, fail to occur.

It has furthermore turned out that in many smokers, apart from the active agent-related, i.e. nicotine-related, physical dependency, there is an additional psychic dependency which cannot be treated by nicotine replacement alone.

This becomes particularly clear when taking into consideration the short half-life of nicotine, which is between 30 minutes and 120 minutes. As a result, smokers should show intense withdrawal symptoms at least in the mornings. Experience shows, however, that the need for a cigarette and the period of time up to smoking the next cigarette often depends strongly on external factors such as stress, sports, company, and the like, and not on real physical symptoms. Hence, tobacco consumption and frequency of consumption can vary considerably depending on a person's psychic constitution.

Often, it is also the psychic dependence which is responsible for the occurrence of relapses.

In this connection, it is worth mentioning that clinical studies have shown that the success rates of smoking cessation can be improved particularly by the combination of nicotine with an antidepressant.

However, the supportive administration of psychopharmacological agents is not without problems due to the side effects and the risk of overdosage or underdosage.

The combination of nicotine or substances with nicotinergic effect with an antidepressant in one pharmaceutical form is desirable because it facilitates intake for the patient and also minimises the risk of application errors.

As smokers in many situations of day to day life feel a need for a cigarette, an application form should be chosen for this type of therapy which guarantees a simple and inconspicuous application and which, if possible, is not reminiscent of the classical pharmaceutical form of the tablet since smoking withdrawal is not a disease in the classical sense, so that it is ensured that the dosage form has good compliance.

In addition, administration to the patient should be accomplished in a manner that is as simple as possible, and the patient should not have any misgivings against taking the medication, for example because of the size of the dosage form or the like. Furthermore, the advantages of the known dosage forms should be avoided.

### SUMMARY OF THE PRESENT INVENTION

It was therefore the object of the present invention to provide nicotine-containing pharmaceutical dosage forms which at the same time enable the administration of an additional active agent, preferably an antidepressant, for combating psychic dependence within a smoking withdrawal therapy. The administration of this additional active agent should exclude side effects to a large extent, and application by the patient should be possible in a simple and reliable way.

It was found that this object is achieved by means of sheet-like dosage forms of a hydrophilic polymer film which disintegrates in the oral cavity and in which at least two active agents have been incorporated, wherein at least one of the active agents is nicotine, a nicotine salt, a nicotine derivative or a substance with nicotinergic action, and wherein at least one further active agent is contained in the film, the further active agent being a member of the group of the psychoactive substances.

### DETAILED DESCRIPTION OF THE PRESENT INVENTION

Accordingly, the dosage forms according to the invention contain a combination of the active agent nicotine, or of a nicotine salt, a nicotine derivative or a substance with nicotinergic action, also summarized under the term of nicotinergic active agents, with at least one further substance which acts on the central nervous system.

The combination of the active agents in the dosage form according to the invention facilitates the intake of both of the active agents for the patient.

In addition, the risk of medication errors is reduced as the patient has to take only one medicament for both of the active agents. This improves compliance and therapy success.

In addition, since specific active agents can be absorbed directly via the mucous membrane, the time until the onset of action occurs is markedly reduced, so that the patient experiences an alleviation of his withdrawal symptoms within an extremely short time.

Through the combination of substances with nicotinergic action, which of course also include nicotine, nicotine salts and nicotine derivatives, with a centrally acting agent, e.g. an antidepressant, it is possible to effectively suppress both the physical and the psychic withdrawal symptoms. In addition, the dosage form according to the invention, as compared to TTS's, offers the advantage that the dose of the active agents can be so low that the person suffering from the withdrawal can apply a dosage form whenever he would reach for a cigarette. In this way, the urge to actively do something against the withdrawal, manifesting itself under normal conditions in the lighting of a cigarette, is likewise satisfied. Satisfying this urge constitutes a component of smoking cessation that is not to be underestimated since smoking used to be connected not only with maintaining the nicotine level, but also with an activity that was perceived as having a relaxing effect.

In addition, when applying the wafer, it produces peak concentrations of nicotine in the blood so that by contrast to the continuous release of nicotine from a TTS with a constant plasma level, a concentration course is obtained which is analogous to that of smoking.

To additionally link the application of the dosage form to a reward effect, taste flavourings or flavouring substances which are perceived to be particularly pleasant may be added to the dosage form.

As the application of the dosage form suppresses the withdrawal symptoms and improves a person's mood, good compliance and optimal efficacy can be ensured.

The administration of these active agent combinations in sheet-like dosage forms (wafers) not only enables easy intake, as explained above, but also an exact tuning of the active agent components, so that false dosages because intake has been forgotten or because of double intake of only one active agent, and consequently an insufficient therapy of a component of the addiction, do not occur.

By varying the ratio of the active agents to each other, it is, in addition, possible to adapt the dosages to the respective needs. Thus, in the course of the nicotine withdrawal the nicotine content can be lowered slowly so that the number of nicotinergic acetylcholine receptors readapts to the normal physiological conditions. Likewise, the dose of antidepressants administered to suppress the psychic dependence may be gradually reduced.

Because of the simple and low-cost manufacture of the wafers, it is possible to provide a large number of medicaments containing different active agent concentrations.

If the wafer is made up of a laminate, it is possible, for example, to alter only the layer thickness of an active agent-containing layer, or to alter the concentration of the active agent.

On the other hand, pharmaceutical products can be produced which have different active agent contents but the same active agent ratio, simply by means of cutting the surface of the dosage form to different sizes.

Furthermore, because of their flat shape, the wafers of the invention, which contain the active agent combinations, can be carried along easily, for example in a wallet, and they are available at once and easy to take, even when travelling.

Water-soluble or swellable polymers that are suitable as a base polymer for the hydrophilic water-soluble and/or swellable polymer film are polymers from the group comprising dextran, polysaccharides, inclusive of starch and starch derivatives, cellulose derivatives, such as carboxymethyl cellulose, ethyl cellulose or propyl cellulose, hydroxypropylmethyl cellulose, hydroxypropyl cellulose, sodium carboxymethyl cellulose (e.g. WALOCEL®), methyl cellulose, hydroxyethyl cellulose and hydroxypropylethyl cellulose, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers. As an alternative, the polymer film may be made of a polyvinyl alcohol-polyethylene glycol graft copolymer.

The proportion of polymer contained in a dosage form according to the invention is preferably 5 to 95%-wt., more preferably 15 to 75%-wt., relative to the dry mass of the dosage form.

The substance acting on the central nervous system which is contained in the dosage forms of the invention in addition to nicotine, is preferably an active agent from the group of the psychopharmacologic agents, which group comprises the active agent groups of the antidepressants, tranquilizers, nootropics, neuroleptics, psychostimulants and psychotomimetics.

Especially preferred are active agents from the group of the antidepressants since they have proved to be highly suitable for overcoming psychic dependence. The invention furthermore encompasses nicotine-containing dosage forms of the type mentioned which contain two or more psychopharmacological agents from the above mentioned active agent groups as additional active agents.

More particularly, the additional substance acting on the central nervous system may be selected from the group which comprises phenothiazines, azaphenothiazines, thioxanthenes, butyrophenones, diphenylbutyl piperidines, iminodibenzyl derivatives, iminostilbene derivatives, dibenzocycloheptadiene derivatives, dibenzodiazepine derivatives, dibenzoxepine derivatives, benzodiazepines, indole derivatives, phenylethylamine derivatives and hypericin derivatives, as well as pharmaceutically acceptable salts or derivatives of these compounds, with the active agent being selected from the group which comprises chlorpromazine, perphenazine, sulpiride, clozapine, risperidone, reserpine, lorazepam, mirtazapine, maprotiline, mianserin, tranylcypromine, moclobemide, oxitriptan, viloxazine, reboxetine, meprobamate, hydroxyzine, buspirone, caffeine, fenetylline, methylphenidate, prolintane, fenfluramine, meclofenoxate, nicergoline, piracetam, pyritinol, as well as pharmacologically acceptable salts of these active agents.

Substances preferably used as antidepressants are brotizolam, triazolam and bupropion.

Substances which may be used as nicotine salts or nicotine derivatives in the dosage forms according to the invention are, preferably, nicotine hydrochloride, nicotine dihydrochloride, nicotine sulfate, nicotine bitartrate, nicotine zinc chloride and nicotine salicylate, either alone or in combination, or in combination with nicotine.

Substances which are preferred as the substances with nicotinergic action, that is, substances, acting at the nicotinic receptor, are, apart from nicotine itself, lobeline, succinylcholine and other peripheral muscle relaxants.

The active substance doses and plasma levels that are suitable for the treatment of psychic dependence are known to those skilled in the art. Preferably, the dose of the substance which acts on the central nervous system is coordinated with the nicotine dose present in the dosage form such that both active agents preferably produce the respective therapeutically beneficial plasma level.

In a preferred dosage form, the pharmaceutical preparation according to the invention contains a combination of two active agents, namely nicotine, a nicotine salt, a nicotine derivative or a substance with nicotinergic action, as well as in addition, as a further active agent component, a substance acting on the central nervous system that is selected from the above-mentioned substances or substance groups.

In another embodiment the pharmaceutical preparation contains two nicotinergic active agents—these active agents may also be nicotine, a nicotinic salt or a nicotine derivative—and one of the above-defined centrally acting substances, with the maximum number of combined active agents not exceeding five.

In another embodiment the pharmaceutical preparation contains a nicotinergic active agent—this may also be nicotine, a nicotine salt or a nicotine derivative—and at least two of the above-defined centrally acting substances, with the maximum number of combined active agents not exceeding five.

The dosage forms according to the invention not only enable nicotine replacement but also allow for the simultaneous treatment of the psychic dependency component of nicotine addiction.

To improve the physicochemical properties, for example reduce the brittleness or embrittlement, humectants, such as glycerine, propylene glycol, sorbitol, mannitol, polyethylene glycol, polyglycerol ester and the like, may be added to the film.

In a further embodiment, antioxidants, for example vitamin C (ascorbic acid), ascorbyl palmitate, vitamin E (tocopherol acetate), hydroxybenzoic acid derivatives, may be added to the wafer, in order to stabilise the film and the active agents. Furthermore, acidic and basic ion exchangers may be used as stabilisers.

In further embodiments, further ingredients such as dyes, pigments, taste flavourings, natural and/or synthetic flavouring substances, sweeteners, buffering systems, may be added to the film. In particular, the taste flavourings and flavouring substances can cover the often bad inherent taste or smell of the active agents and/or give the dosage form a pleasant taste, so that the patient's readiness to take the medication is considerably improved.

The addition of buffering systems on the one hand serves to stabilise the film and the active agents against outside influences and during storage; on the other hand, the pH of the dosage form can thereby be adjusted to a physiologically acceptable pH value, so that irritation of mucous membranes is avoided. By using a buffering system, it is also possible to improve the solubility of acidic or basic active agents in the matrix.

The dosage forms according to the invention are configured so as to be thin, for example in the form of a wafer. The thickness of the dosage form is preferably 0.1 to 5 mm, more preferably 0.5 to 1 mm. The lower limit for the thickness of the dosage form is about 50 μm. The surface area of the dosage form is between 0.09 cm² and 12 cm², preferably between 1 cm² and 8 cm², and more preferably between 3 cm² and 6 cm².

In a further embodiment, the wafers of the present invention contain a disintegrant or a wicking agent, for example a bicarbonate-acid mixture or an aerosil, being activated by contact with a liquid and accelerating the disintegration of the wafer after application thereof, and thereby also accelerating the release of active agent.

In a preferred embodiment, the wafer is present as a foam so that the release of active agent takes place even more rapidly because of the enlarged surface. In this embodiment, the cavities of the foam may contain one or more of the active agents in liquid form.

To improve the absorption of the active agents via the mucous membrane, permeation enhancers, such as substances from the groups of the fatty alcohols, fatty acids, poly-oxyethylene fatty alcohol ethers, polyoxyethylene fatty acid esters, fatty alcohol esters and fatty acid esters, particularly sorbitan monolaurate or esters of long-chain fatty acids with methyl, ethyl or isopropyl alcohol, or esters of fatty alcohols with acetic acid or lactic acid, or substances such as DMSO (dimethyl sulfoxide) and oleic acid diethanolamine may likewise be incorporated in the film. The constituent amount of these substances is 0.1 to 25%-wt., preferably 1 to 10%-wt., in each case relative to the total weight of the active agent matrix.

Furthermore, the composition of the wafer may contain compounds that retard the release of active agent (e.g., microencapsulation).

In a further embodiment, the wafer has mucoadhesive properties, so that it adheres to the mucous membrane until it is completely dissolved.

In a preferred embodiment, at least one of the active agents is bound to an ion exchanger, so that the hydrophilic polymer disintegrates quickly in the oral cavity, whereas the release of active agent is retarded or occurs when the pH has changed, e.g. in the gastrointestinal tract. In this way, active agents having a different mechanism of action and absorption can be administered in one dosage form, that is, at least one of the released active agents is either absorbed at the site of application, for example via the mucous membrane, or it is transported further and absorbed at another site.

The wafer may also be made up as a laminate with different layers, with the active agents being contained in discrete layers which are spatially separated from each other and differ from each other in terms of their composition. In this way, the active agents can be released at different sites of action, but also with retardation, if the disintegration times of the various layers of the wafer differ from each other.

Likewise, the active agents may be arranged within layers that disintegrate at different rates, so that the preparation as a whole shows a retardation effect.

In a further embodiment, one of the outer layers may be mucoadhesive, to promote the adherence of the dosage form on the mucous membrane and to facilitate the active agent absorption via the mucous membrane by establishing direct contact.

The disintegration of the inventive dosage form in an aqueous medium preferably takes place in the range from 1 second to 5 minutes, more preferably in a range from 5 seconds to 1 minute, and most preferably in the range from 10 seconds to 30]] seconds.

The dosage forms according to the invention are advantageously suitable for administering medicaments in the oral cavity or for rectal, vaginal or intranasal administration. They can be used in human medicine as well as in veterinary medicine.

The present invention furthermore relates to the use of an active agent combination according to the invention for the production of an oral dosage form for smoking withdrawal, said dosage form preferably being formulated as a wafer.

Furthermore, the present invention relates to a method for therapeutic smoking withdrawal, wherein the administration of an above-described active agent combination of nicotine and a centrally acting substance is carried out by means of an orally applicable dosage form with transmucosal absorption.

Finally, the present invention also relates to a method for the production of a sheet-like dosage form, comprising the following steps:
preparing a solution containing at least one polymer and at least two active agents, of which one is nicotine, a nicotine salt, a nicotine derivative, or a substance with nicotinergic action, and the other one is a psychopharmacological agent;
spread-coating the solution on a coating substrate; and
solidifying the spread-coated solution by drying and withdrawing the solvent.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A sheet-like pharmaceutical preparation based on least one hydrophilic polymer and including an active agent combination, said polymers rapidly disintegrating upon contact with moisture and for releasing the active agent combination for smoking withdrawal, wherein said active agent combination comprises at least two active agents, wherein at least one of said at least two active agents is selected from the group consisting of the nicotinergic active agents, and wherein at least one of said at least two active agents is selected from the group consisting of brotizolam, triazolam, bupropion, lorazepam, mirtazapine and reboxetine.

2. The pharmaceutical preparation according to claim 1, wherein the group of the nicotinergic active agents is selected from the group consisting of nicotine, nicotine derivatives, the corresponding pharmaceutically acceptable salts of nicotine and nicotine derivatives compounds with nicotinergic action.

3. The pharmaceutical preparation according to claim 1, further comprising a further active agent selected from the group consisting of the psychopharmacological agents.

4. The pharmaceutical preparation according to claim 3, wherein said psychopharmacological agents are selected from the group consisting of the antidepressants, tranquilisers, nootropics, neuroleptics, psychostimulants and psychotomimetics.

5. The pharmaceutical preparation according to claim 3, wherein at least one of the active agents present in addition to nicotine is selected from the group consisting of phenothiazines, azaphenothiazines, thioxanthenes, butyrophenones, diphenylbutyl piperidines, iminodibenzyl derivatives, iminostilbene derivatives, dibenzocycloheptadiene derivates, dibenzodiazepine derivatives, dibenzoxepine derivatives, benzodiazepine, indole derivatives, phenylethylamine derivatives and hypericin derivatives, as well as pharmaceutically acceptable salts or derivatives of these compounds.

6. The pharmaceutical preparation according to claim 3, wherein at least one of the active agents present in addition to nicotine is selected from the group consisting of chlorpromazine, perphenazine, sulpiride, clozapine, risperidone, reserpine, maprotiline, mianserin, tranylcypromine, moclobemide, oxitriptan, viloxazine, meprobamate, hydroxyzine, buspirone, caffeine, fenetylline, methylphenidate, prolintane, fenfluramine, meclofenoxate, nicergoline, piracetam, pyritinol, as well as their pharmacologically acceptable salts.

7. The pharmaceutical preparation according to claim 1, wherein the nicotine salts and the nicotine derivatives are selected from the group consisting of nicotine hydrochloride, nicotine dihydrochloride, nicotine sulfate, nicotine bitartrate, nicotine zinc chloride and nicotine salicylate, as well as combinations of these compounds.

8. The pharmaceutical preparation according to claim 2, wherein the substances with nicotinergic action are selected from the group consisting of nicotine, lobeline, succinylcholine and other peripheral muscle relaxants, as well as combinations of these substances.

9. The pharmaceutical preparation according to claim 1, wherein the at least one hydrophilic polymer is selected from the group consisting of dextran, polysaccharides, inclusive of starch and starch derivatives, cellulose derivatives, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers.

10. The pharmaceutical preparation according to claim 1, wherein the at least one hydrophilic polymer is a polymer film comprising a polyvinyl alcohol-polyethylene glycol graft copolymer.

11. The pharmaceutical preparation according to claim 1, wherein the preparation further comprises a humectant selected from the group consisting of glycerine, propylene glycol, sorbitol, mannitol, polyethylene glycol and polyglycerol ester.

12. The pharmaceutical preparation according to claim 1, wherein the preparation further comprises an antioxidant selected from the group consisting of vitamin C (ascorbic acid), ascorbyl palmitate, vitamin E (tocopherol acetate) and hydroxybenzoic acid derivatives.

13. The pharmaceutical preparation according to claim 1, wherein the active agent of the preparation is bound to an acidic or basic ion exchanger for taste masking.

14. The pharmaceutical preparation according to claim 1, wherein the preparation further comprises dyes and/or pigments.

15. The pharmaceutical preparation according to claim 1, wherein the preparation further comprises natural and/or synthetic flavouring substances.

16. The pharmaceutical preparation according to claim 1, wherein the preparation further comprises a disintegrant or a wicking agent.

17. The pharmaceutical preparation according to claim 1, further comprising a buffer system for adjusting the pH value of the preparation.

18. The pharmaceutical preparation according to claim 1, wherein the at least one hydrophilic polymer disintegrates within less than 5 minutes after application in the oral cavity of the user.

19. The pharmaceutical preparation according to claim 1, wherein the at least one hydrophilic polymer disintegrates quickly in the oral cavity whereas the active agent remains bound to an ion exchanger which releases said active agent only upon reaching the gastrointestinal tract.

20. The pharmaceutical preparation according to claim 1, wherein the active agents are contained in discrete layers which are spatially separated from each other and which differ from each other in terms of the respective compositions.

21. The pharmaceutical preparation according to claim 1, wherein the preparation is present as a foam having cavities and at least one of the active agents is present in liquid form within the cavities of said foam.

22. The pharmaceutical preparation according to claim 1, wherein said preparation contains a combination of a nicotinergic active agent and an antidepressant.

23. Use of a dosage form according to claim 1 for rectal, vaginal or intranasal administration of pharmaceutical active agents to humans or animals.

24. Use of the pharmaceutical preparation according to claim 1, the active agent combination comprises a combination of nicotinergic active agent and a psychopharmacological agent for the producing an oral dosage form for smoking withdrawal.

25. Use of the pharmaceutical preparation according to claim 1, the active agent combination comprises a combination of nicotinergic active agent and an antidepressant for producing an oral dosage form for smoking withdrawal.

26. The use according to claim 23, wherein the pharmaceutical preparation is formulated as a wafer.

27. A method for the therapeutic treatment of a person suffering from withdrawal symptoms caused by smoking withdrawal, comprising the step of orally administering an orally applicable dosage form with transmucosal absorption comprising an active agent combination of nicotinergic active agent and psychopharmacological agent, said dosage form being based on hydrophilic polymers, the polymers rapidly disintegrating upon contact with moisture.

28. A method for producing a sheet-like dosage form based on at least one hydrophilic polymers and including an active agent combination, the polymers rapidly disintegrating upon contact with moisture for releasing an active agent combination for smoking withdrawal, wherein the active agent combination comprises at least two active agents, wherein at least one of said at least two active agents is selected from the group consisting of the nicotinergic active agents, and wherein at least one of said at least two active agents is selected from the group consisting of brotizolam, triazolam, bupropion, lorazepam, mirtazapine and reboxetine, said method comprising the steps of:
preparing a solution which contains at least one polymer and at least two active agents, one of said active agents being selected from the group consisting of nicotine, a nicotine salt, a nicotine derivative and a substance with nicotinergic action, and the other of said active agents being a psychopharmacological agent selected from the group consisting of brotizolam, triazolam, bupropion, lorazepam, mirtazapine and reboxetine;
spread-coating the solution on a coating substrate; and
solidifying the spread-coated solution by drying and withdrawing the solvent.

29. The pharmaceutical preparation according to claim 19, wherein said cellulose derivatives are selected from the group consisting of carboxymethyl cellulose, ethyl cellulose or propyl cellulose, hydroxypropylmethyl cellulose, hydroxypropyl cellulose, sodium carboxymethyl cellulose, methyl cellulose, hydroxyethyl cellulose and hydroxypropylethyl cellulose.

30. The pharmaceutical preparation according to claim 18, wherein the hydrophilic polymer disintegrates within less than 3 minutes after application in the oral cavity.

31. The pharmaceutical preparation according to claim 30, wherein the hydrophilic polymer disintegrates within less than 1 minute after application in the oral cavity.

32. The pharmaceutical preparation according to claim 31, wherein the hydrophilic polymer disintegrates within less than 30 seconds, after application in the oral cavity.

33. The use according to claim 24, wherein the pharmaceutical product is formulated as a wafer.

34. The use according to claim 25, wherein the pharmaceutical product is formulated as a wafer.
